(19) United States
(12) Patent Application Publication
(10) Pub. No.: US 2009/0210967 A1
(43) Pub. Date: Aug. 20, 2009

(54) Glutamate Receptors as Regulators of Carbon Transport, Mobilization, Distribution, Reallocation, and Partitioning in Higher Plants

(76) Inventors: Frank J. Turano, Baltimore, MD (US); Jiman Kang, Falls Church, VA (US); John Quackenbush, Dover, MA (US); Jason Shockey, Severna Park, MD (US); Chen Zeng, Rockville, MD (US)

Correspondence Address: BLANK ROME LLP, WATERGATE, 600 NEW HAMPSHIRE AVENUE, N.W., WASHINGTON, DC, 20037, US

(21) Appl. No.: 12/252,573

(22) Filed: Oct. 16, 2008

Related U.S. Application Data
(60) Provisional application No. 60/980,449, filed on Oct. 17, 2007.

(51) Int. Cl. C12N 15/82; A01H 5/00; C12N 15/63

(52) U.S. Cl. 800/286; 800/298; 435/468; 435/320.1

(57) ABSTRACT

This invention relates to compounds for improving plant growth and characteristics, improved modified plants, processes for obtaining the same, and improved methods of obtaining plant products, and specifically those concerning AtGLR1.1.

### CROSS REFERENCE TO RELATED APPLICATIONS

This application claims benefit of the filing date and right of priority under 119(e) to U.S. 60/980,449, the contents of which are incorporated herein in their entirety.

### STATEMENT REGARDING FEDERALLY SPONSORED RESEARCH OR DEVELOPMENT

No federal government funds were used in researching or developing this invention.

Names of Parties to a Joint Research Agreement

The George Washington University

The Institute for Genomic Research

Reference to a Sequence Listing

A table or a computer list appendix on a compact disc

[ ] is

[ ] is not

included herein and the material on the disc, if any, is incorporated-by-reference herein.

### BACKGROUND

Field of the Invention

This invention relates to compounds for improving plant growth and characteristics, improved modified plants, processes for obtaining the same, and improved methods of obtaining plant products.

Background of the Invention

The current state of knowledge is as follows. The transcription factor, MYB61, that has been shown to be is necessary and sufficient for the remobilization of carbon into lignin, and it is know that MYB61 mediated lignin biosynthesis is stimulated in det3 mutants in the dark grown seedlings via a AtGLR (Dubos et al. 2005. Plant J. 43:348-355), the control and regulation of the process was not know to be specifically modulated by AtGLR1.1. Here we show that silencing of AtGLR1.1 stimulates lignin biosynthetic genes. Prior to this work it was not known which of the twenty AtGLRs actually regulated MYB61 and lignin biosynthesis.

Likewise, it has been postulated that carbon is remobilization into starch via an AtGLR-mediated mechanism (Dubos et al. 2005. Plant J. 43:348-355). Here we show that silencing of AtGLR1.1 stimulates starch biosynthetic genes and starch biosynthesis. Prior to this work it was known which of the twenty AtGLRs actually regulated C remobilization into starch.

The MYB transcription factors PAP1, PAP2 and MYB123 have been shown to control anthocyanin biosynthesis. Here we show that silencing of AtGLR1.1 stimulates PAP 1, PAP2 expression and expression of the genes in the anthocyanin biosynthetic pathway as well as anthocyanin accumulation.

It is known that phosphoenolpyruvate carboxylase kinase (PPCK1) phosphorylates phosphoenolpyruvate carboxylase (PEPC) that catalyzes the synthesis of OAA from phosphoenolpyruvate (PEP) and bicarbonate. The constitutive over-expression of a positive dominant PEPCK construct in Arabidopsis redirected carbon from sugars into organic acids and amino acids. Here we show that AtGLR1.1 regulates this process because the down regulation of AtGLR1.1, results in the opposite affect: that is OAA is reallocated from amino acid biosynthesis into lipid.

### BRIEF SUMMARY OF THE INVENTION

The novel component of this work identifies the AtGLR or plant glutamate receptor as carbon sensors and regulators of carbon mobilization, allocation and partitioning,

The invention described here demonstrates that the AtGLRs function to regulate carbon metabolism and carbon partitioning, reallocation, redistribution, sensing and they modulate growth and development and will affect nitrogen, sulfur and phosphate, sensing, up-take, distribution, assimilation, partitioning and allocation.

The present invention can be used to:

Develop plants with higher lignin, lignin biosynthesis, biomass, growth and yield;

Develop plants with higher modified in cell wall composition;

Develop plants with higher sucrose levels;

Develop plants with higher oil or lipid content; and

Develop plants with altered amino acid content

The present invention relates to polynucleotides and polypeptides that may be used to improve or modify plant carbon metabolism, allocation, distribution, reallocation, redistribution, partitioning and assimilation. More specifically, this invention is related to the role of Arabidopsis thaliana glutamate receptors (AtGLRs) in the regulation of the biosynthesis, metabolism, catabolism, transport and mobilization of carbon metabolites, carbohydrates and carbon-based polymers, and the utilization of the AtGLRs to alter carbon-based signaling molecules, growth regulators, structural compounds to control and improve plant growth, development, yield and crop quality.

This invention describes the use of plant glutamate receptors (GLRs) to reallocate carbon metabolites in higher plants or to alter the accumulation or distribution of carbon metabolites or compounds such as sugars, organic acids, carbohydrates, starch, oils, lipids, callose, cellulose, hemicellulose and secondary compounds such as anthocyanins, flavonoids and lignins in plants (Paul and Pellny 2003, J. Exper. Bot. 54:539-547). Increased levels of these compounds are useful for the development of food chemistry, biofuels, oils, fiber, wood, and plant protectants.

Accordingly, provided herein in a preferred embodiment is a method of delivering to a plant target cell a DNA that is expressed in the target cell comprising administering a vector to the target cell, wherein said vector transduces the target cell; and wherein said vector has been modified to comprise a DNA which comprises an expressible gene and said gene is expressed in said target cell either constitutively or under regulatable conditions, and wherein the expressible gene encodes a messenger RNA which is antisense with respect to a messenger RNA transcribed from a gene endogenous to said cell, and wherein the antisense RNA is antisense to the mRNA which is translatable into a glutamate receptor.

In another preferred embodiment is provided the method wherein the glutamate receptor is Arabidopsis thaliana glutamate receptor 1.1 (AtGLR1.1).

In another preferred embodiment is provided a method of regulating plant metabolism, comprising: delivering to a plant target cell a DNA that is expressed in the target cell comprising administering a vector to the target cell, wherein said vector transduces the target cell; and wherein said vector has been modified to comprise a DNA which comprises an expressible gene and said gene is expressed in said target cell either constitutively or under regulatable conditions, and wherein the expressible gene encodes a messenger RNA which is antisense with respect to a messenger RNA transcribed from a gene endogenous to said cell, and wherein the antisense RNA is antisense to the mRNA which is translatable into a glutamate receptor.

In another preferred embodiment is provided the method wherein the antisense RNA is antiGLR and is capable of altering carbon-based signaling molecules, growth regulators, structural compounds to control and improve plant growth, development, yield and crop quality.

In another preferred embodiment is provided the method wherein the antisense RNA is antiGLR and is capable of reallocating carbon metabolites in higher plants and altering the accumulation or distribution of carbon metabolites or compounds, wherein said metabolites or compounds comprise sugars, organic acids, carbohydrates, starch, oils, lipids, callose, cellulose, hemicellulose and secondary compounds including anthocyanins, flavonoids and lignins.

In another preferred embodiment is provided the method wherein the glutamate receptor is Arabidopsis thaliana glutamate receptor 1.1 (AtGLR1.1).

In another preferred embodiment is provided a DNA vector comprising an expressible gene that is expressed in a target plant cell, wherein the expressible gene encodes a messenger RNA which is antisense with respect to a messenger RNA transcribed from a gene endogenous to said cell, and wherein the antisense RNA is antisense to the mRNA which is translatable into a glutamate receptor.

In another preferred embodiment is provided, a plant comprising the DNA vector described herein.

### BRIEF DESCRIPTION OF THE DRAWINGS

FIG. 1A and 1B are a series of protein gel electrophoresis results.

FIG. 2A, 2B, 2C, and 2D are gene expression charts.

FIG. 3 is a series of bar graphs showing sugar, starch and anthocyanin results.

FIG. 4 is a bar graph of amino acids in antiAtGLR1.1 versus wild type.

FIG. 5 is a bar graph of the expression (log₂ ratio) of various mRNAs or transcripts.

### DETAILED DESCRIPTION OF THE INVENTION

Definitions

The following definitions are provided as an aid to understanding the detailed description of the present invention.

The phrases “coding sequence,” “coding region,” “structural sequence,” and “structural nucleic acid sequence” refer to a physical structure comprising an orderly arrangement of nucleotides. The nucleotides are arranged in a series of triplets that each form a codon. Each codon encodes a specific amino acid. Thus, the coding sequence, coding region, structural sequence, and structural nucleic acid sequence encode a series of amino acids forming a protein, polypeptide, or peptide sequence. The coding sequence, coding region, structural sequence, and structural nucleic acid sequence may be contained within a larger nucleic acid molecule, vector, or the like. In addition, the orderly arrangement of nucleotides in these sequences may be depicted in the form of a sequence listing, figure, table, electronic medium, or the like.

The phrases “DNA sequence,” “nucleic acid sequence,” and “nucleic acid molecule” refer to a physical structure comprising an orderly arrangement of nucleotides. The DNA sequence or nucleotide sequence may be contained within a larger nucleotide molecule, vector, or the like. In addition, the orderly arrangement of nucleic acids in these sequences may be depicted in the form of a sequence listing, figure, table, electronic medium, or the like.

The term “expression” refers to the transcription of a gene to produce the corresponding mRNA and translation of this mRNA to produce the corresponding gene product (i.e., a peptide, polypeptide, or protein).

The phrase “expression of antisense RNA” refers to the transcription of a DNA to produce a first RNA molecule capable of hybridizing to a second RNA molecule, said second RNA molecule encodes a gene product that is desirably down-regulated.

The term “homology” refers to the level of similarity between two or more nucleic acid or amino acid sequences in terms of percent of positional identity (i.e., sequence similarity or identity). Homology also refers to the concept of similar functional properties among different nucleic acids or proteins.

The term “heterologous” refers to the relationship between two or more nucleic acid or protein sequences that are derived from different sources. For example, a promoter is heterologous with respect to a coding sequence if such a combination is not normally found in nature. In addition, a particular sequence may be heterologous with respect to a cell or organism into which it is inserted (i.e., does not naturally occur in that particular cell or organism).

The term “hybridization” refers to the ability of a first strand of nucleic acid to join with a second strand via hydrogen bond base pairing when the two nucleic acid strands have sufficient sequence complementarity. Hybridization occurs when the two nucleic acid molecules anneal to one another under appropriate conditions.

The terms “plants” and “plant”, in the context of the present invention, refer to higher plants.

The phrase “operably linked” refers to the functional spatial arrangement of two or more nucleic acid regions or nucleic acid sequences. For example, a promoter region may be positioned relative to a nucleic acid sequence such that transcription of the nucleic acid sequence is directed by the promoter region. Thus, a promoter region is operably linked to the nucleic acid sequence.

The terms “promoter” or “promoter region” refers to a nucleic acid sequence, usually found upstream (5′) to a coding sequence, which is capable of directing transcription of a nucleic acid sequence into mRNA. The promoter or promoter region typically provides a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, and the like. The activity or strength of a promoter may be measured in terms of the amounts of RNA it produces, or the amount of protein accumulation in a cell or tissue, relative to a second promoter that is similarly measured.

The term “5′-UTR” refers to the untranslated region of DNA upstream, or 5′, of the coding region of a gene.

The term “3′-UTR” refers to the untranslated region of DNA downstream, or 3′, of the coding region of a gene.

The phrase “recombinant vector” refers to any agent by or in which a nucleic acid of interest is amplified, expressed, or stored, such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source and is capable of genomic integration or autonomous replication.

The phrase “regulatory sequence” refers to a nucleotide sequence located upstream (5′), within, or downstream (3′) with respect to a coding sequence. Transcription and expression of the coding sequence is typically impacted by the presence or absence of the regulatory sequence.

The phrase “substantially homologous” refers to two sequences that are at least about 90% identical in sequence, as measured by the CLUSTAL W method in the Omiga program, using default parameters (Version 2.0; Accelrys, San Diego, Calif.).

The term “transformation” refers to the introduction of nucleic acid into a recipient host. The term “host” refers to bacteria cells, fungi, animals or animal cells, plants or seeds, or any plant parts or tissues including plant cells, protoplasts, calli, roots, tubers, seeds, stems, leaves, seedlings, embryos, and pollen.

As used herein, the phrase “transgenic plant” refers to a plant having an introduced nucleic acid stably introduced into a genome of the plant, for example, the nuclear or plastid genomes.

As used herein, the phrase “substantially purified” refers to a molecule separated from substantially all other molecules normally associated with it in its native state. More preferably, a substantially purified molecule is the predominant species present in a preparation. A substantially purified molecule may be greater than about 60% free, preferably about 75% free, more preferably about 90% free, and most preferably about 95% free from the other molecules (exclusive of solvent) present in the natural mixture. The phrase “substantially purified” is not intended to encompass molecules present in their native state.

As used herein, “variants” have substantially similar or substantially homologous sequences when compared to reference or wild type sequence. For nucleotide sequences that encode proteins, “variants” also include those sequences that, because of the degeneracy of the genetic code, encode the identical amino acid sequence of the reference protein. Variant nucleic acids also include those that encode polypeptides that do not have amino acid sequences identical to that of the proteins identified herein, but which encode an active protein with conservative changes in the amino acid sequence.

Plant Metabolism

Carbon is critical for many aspects of plant metabolism. Carbon is a basic building block for sugars (mono-, di- and polysaccharides), organic acids, carbohydrates, starch, cellulose, hemi-cellulose, callose, pectin and lignins (Paul and Pellny 2003, J. Exper. Bot 54:539-547). Sugars can function as important signaling molecules in plants (Rolland et al. 2002. Plant Cell 14:S185-205) and are required for nucleotide, starch, callose, cellulose, pectin and hemi-cellulose biosyntheses. Carbon pathways such as the pentose phosphate shunt and the Calvin cycle provide reductant (energy) for many biological processes, such as nitrogen and sulfur up-take, assimilation, and metabolism. Carbon, in the form of organic acids, function as buffers, chelators, key components of energy cycles (Calvin and Kreb cycles) that are involved in photosynthesis and the generation of energy in mitochondria. Organic acids also provide backbones or skeletons for amino acid biosynthesis and are intimately linked with the assimilation of nitrogen into amino acids (Chen and Gadal 1990. Plant Physiol. Biochem. 28:141-145; Lancien et al. 2000. Plant Physiol. 123: 817-824; Hodges 2002. J. Exper. Bot. 53, 905-991) and may have signaling properties (Lancien et al. 2000. Plant Physiol. 123: 817-824; Hodges 2002. J. Exper. Bot. 53, 905-991). Carbon assimilation, transport, allocation, redistribution and sequestration are important to many aspects of agriculture, food industry, forestry, and horticulture and fiber production. Furthermore, as the levels of CO₂ are expected to continue to rise, this invention could provide alternatives for the sequestration of CO₂ and thus could be a viable solution to the Greenhouse Gas problem.

Hexokinase has been proposed to be a glucose sensor that controls several developmental and stress-related processes in plants (Jang et al. 1997. Plant Cell 9, 5-19; Sheen et al. 1999. Curr Opin. Plant Biol. 2: 410-408). Although hexokinase has been proposed to be the glucose sensor. Although hexokinase is the glucose sensor its role in carbon allocation, partitioning, or distribution, has not been established. Likewise the receptors or sensors for other carbon compounds carbohydrate or organic acids have not been identified (Smeekens. 2000. Ann. Rev. Plant Physiol. Plant Mol. Boil. 51:49-81; Polge & Thomas 2006. Trens Plant Sci. 12:20-28). The proteins and genes that control and regulate carbon partitioning, allocation and distribution have not been identified or established. Components of the carbon signaling and sensing system, or matrix, is hypothesized to be part of an integrated system that is connected with other assimilatory and metabolic pathways or networks (Coruzzi & Zhou 2001. Curr. Opin. Plant Biol. 4, 247, Coruzzi & Bush 2001. Plant Physiol. 125, 61). The mechanism(s) that control carbon metabolism are intimately linked with those that control the up-take, assimilation, transport, mobilization and metabolism of other elements required for plant growth and metabolism, i.e. nitrogen, sulfur (, and phosphate (Plaxton and Carswell 1999. In: HR Lerner, ed. Plant responses to environmental stresses: from phytohormones to genome reorganization. Pp. 349-372. Nielsen et al. 2001. Exp Bot 52: 329-339:Wu et al. 2003. Plant Physiol. 132, 1260-1271; Jain et al. 2007 Plant Physiol 16, on line asDOI:10.1104/pp. 106.092130). The sensing and subsequent reallocation or redistribution of carbon may have wide ramifications and extend to improvements in nitrogen-use-efficiency and utilization of nitrogen and sulfur, metabolism and accumulation of amino acids and secondary metabolites that are composed of these compounds, such as anthocyanins (Deikman and Hammer, 1995 Plant Physiol. 108: 47-57; Martin et al., 2002; Mita et al., 1997. Plant J. 11: 841-851; Tsukaya et al., 1991. Plant Physiol. 97: 1414-1421), flavonoids (Nikiforova et al., 2003. Plant J. 33: 633-650), and glucosinolates (Bones and Rossiter 1996. Physiol. Plant. 97: 194-208). These metabolic changes may result in physiological improvements and may positively affect water-use-efficiency (Martin et al. 1999. Crop Science 39:1775-1783), biomass (Martin et al. 1999. Crop Science 39:1775-1783), fiber quality (Pettigrew 2001 Crop Science 41:1108-1113) and wood production (Myneni 2001. PNAS 98, 14784-14789), and affect plant growth and development and crop productivity and quality (Mokhtari et al. 2006. J. Food Agricul. Environ. 4: 288-294).

In animals the ionotropic glutamate receptors (iGLRs) control signaling across a small gap between adjacent neurons called the synapse (Madden DR 2002. Nat. Rev. Neurosci.3:91-101). Plants, such as Arabidopsis (Lam et al. 1998. Nature 396:125-126) and rice and some bacteria, such as Synechosystis PCC 6803 (Chen et al. 1999. Nature 402:817-821) also contain putative iGLR homologs. It has been suggested that these receptors function in a primitive well-conserved sensing system that existed before animals, and components in the system may have evolved into neuronal signaling in higher animals.

There are twenty iGLRs homologs in the plant Arabidospsis, designated the Arabidopsis thaliana glutamate receptors (AtGLRs). The 20 AtGLRs separate into three distinct phylogenetic groups, or clades, called AtGLR1, AtGLR2 and AtGLR3. Specific AtGLRs are designated by the clade number followed by the “member” number, i.e. the first member of clade 3 is AtGLR3.1 (Lacombe et al. 2001. Science. 292: 1486-1487). AtGLRs are structurally similar to the animal N-methyl-D-aspartate (NMDA)-type iGLRs (Davenport 2002. Ann. Bot. 90: 549-557) that control neural signaling (Meldrum 2000. J. Nutr. 130; 1007S-1015S). The mammalian N-methyl-D-aspartate (NMDA)-type iGLRs have two potential binding domains (BDs). One in the amino terminal domain (ATD) that contains a region called the leucine-isoluecine-valine-binding-protein (LIVBP)-like domain (LIVBP-LD) that binds modulators (Zheng et al. 2001. Nat. Neurosci. 4;894-901; Huggins and Grant 2005. J. Mol. Graph. Model. 23; 381-388) and functions in dimerization and receptor assembly (Perez-Otano et al. 2001. J. Neurosci. 21;1228-1237. The other BD, the lysine-arginine-ornithine-binding-protein (LAOBP)-like domain (LAOBP-LD), binds the ligand, or agonist(s), to activate the receptor (Mayer and Armstrong 2004. Ann. Rev. Physiol. 66:161-181: Mayer 2006. Nature 440:456-462). Plus there are the three (1, 2 and 3) transmembrane (TM) and one pore forming (P), domains.

The expression of all twenty AtGLRs has been determined in Arabidopsis organs i.e. leaves, roots, flowers, or siliques (Chiu et al. 2002. Mol. Biol. Evol. 19:1066-1082) and their phylogenetic relationship to bacterial and animal GLRs has been well documented (Chiu et al. 1999. Mol. Biol. Evol. 16, 826-838; Chiu et al. 2002. Mol. Biol. Evol. 19:1066-1082; Turano et al. 2001. Mol. Biol. Evol. 18:1417-1420). Physiological analyses show that these receptors are involved in the regulation of carbon and nitrogen metabolism (Kang and Turano. 2003. PNAS. 100:6872-6877), carbon allocation (Dubos et al. 2005. Plant J. 43:348-355), calcium homeostasis (Kim et al. 2001. Plant Cell Physiol. 42:74-84), and stress responses (Meyerhoff et al. 2005. Planta. 222:418-27), starch and lignin biosyntheses (Dubos et al. 2005. Plant J. 43:348-355).

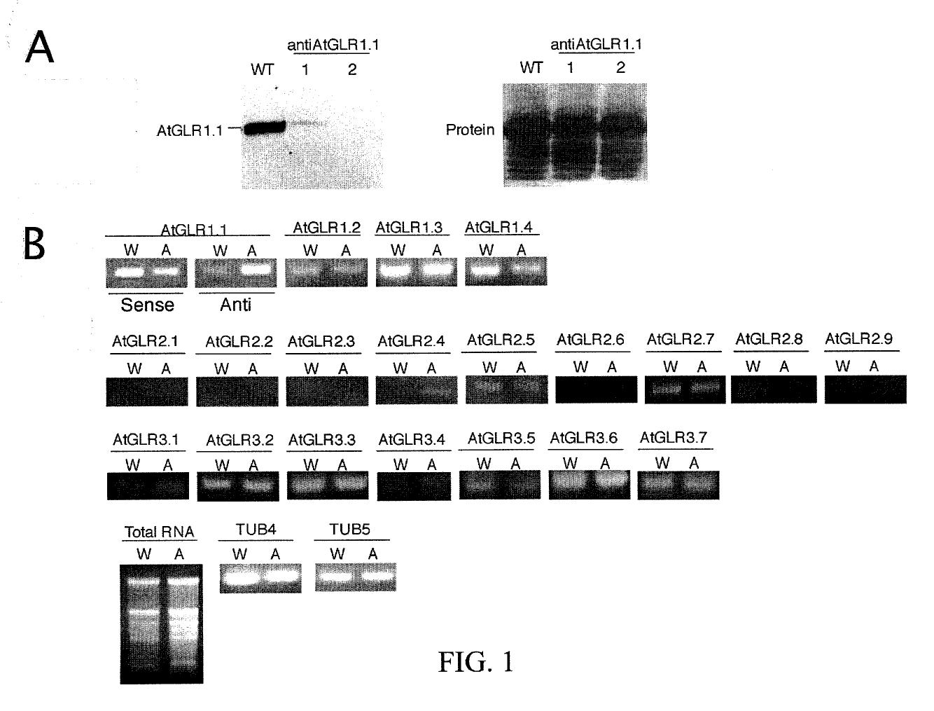

Antisense directed at AtGLR1.1 (antiAtGLR1.1) resulted in a decrease in the AtGLR1.1 peptide (FIG. 1A), this decrease in protein was shown previously (Kang and Turano. 2003. PNAS. 100:6872-6877) and semi-quantitative RT-PCR (FIG. 1B) showed that the endogenous AtGLR1.1 transcript (Sense) is significantly lower (˜65%) in antiAtGLR1.1 than in WT lines, due to the over-expression of the antisense AtGLR1.1 (Anti) transcript. The difference between the observed levels of the AtGLR1.1 transcript (˜35%) and AtGLR1.1 peptide (˜0%) in antiAtGLR1.1 versus WT lines is due to translational suppression of N-related peptides in antiAtGLR1 lines as previously reported (Kang and Turano. 2003. PNAS 100:6872-6877). RT-PCR analysis showed no detection of transcripts for AtGLR2.1, 2.2, 2.3, 2.6, 2.8 and 2.9 in WT or antiAtGLR1.1 plants. Eleven AtGLR transcripts were readily detected in leaves (1.1, 1.2, 1.3, 1.4, 2.5, 2.7, 3.2, 3.3, 3.5, 3.6 and 3.7), and three other AtGLRs (2.4, 3.1 and 3.4) had appreciable accumulation in both plants. These findings are similar to those observed by Chiu et al. (Chiu et al. 2002. Mol. Biol. Evol. 19:1066-1082). The data show that AtGLR1.1 has been “silenced” by using an antisense approach (FIG. 1).

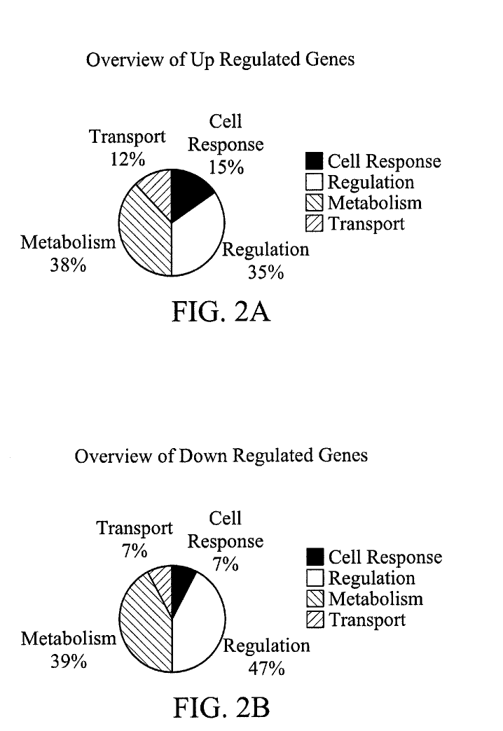

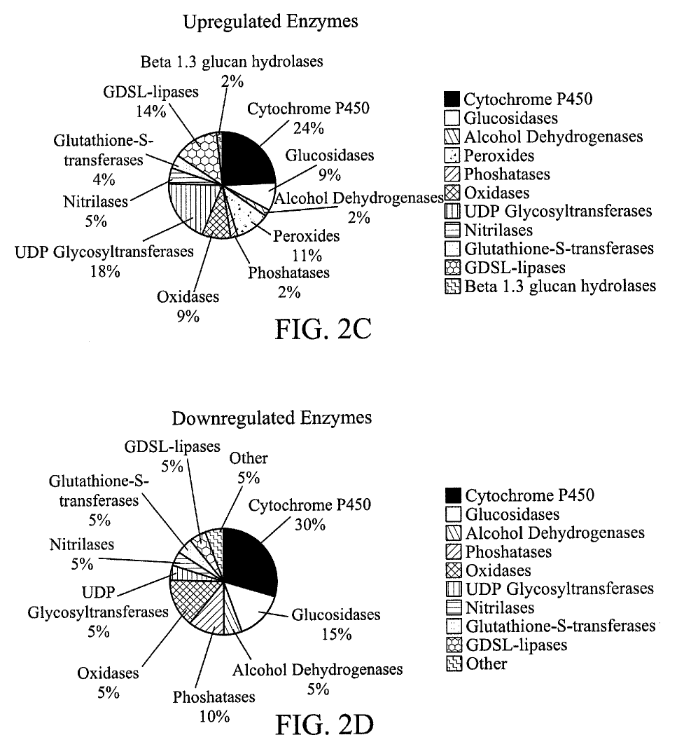

To elucidate the role of antiAtGLR1.1 in the regulation of metabolic networks, an Arabidopsis genome-wide microarray analysis was used to determine the difference in transcript accumulation between two independently transformed homozygous antiAtGLR1.1 and WT lines. 876 genes were shown to have different expression patterns by performing a t-test (P<0.05) and average log ratios greater than 0.6 or less than —0.6 were retained in the dataset. The false discovery rate of the 876 genes, determined by Significance Analysis of Microarrays (SAM) was 0.013. There was a significant increase in 533 transcripts, where as there was a significant decrease in 343 transcripts in antiAtGLR1.1 versus WT lines (Table 1), the data were categorized according to Usadel et al. into functional gene classes and large gene families (FIG. 2) and in to metabolic and biosynthetic pathways using the Metabolic Map in AraCyc at TAIR (http://www.arabidopsis.org: 1555/ARA/new-image?type=OVERVIEW) and MAPMAN (Usadel et al. 2005. Plant Physiol. 138: 1195-1204). The accumulation of several carbon-related transcripts (Table 2) was significantly (P-value<0.05) altered in leaves of 30-d-old antiAtGLR1.1 versus WT plants.

### Sucrose Metabolism and Redistribution

Transcripts of several sucrose (Suc)-metabolic genes significantly increase in antiAtGLR1.1 lines (Table 2). A Suc synthase (Sus) transcript (At5g20830), that encodes a key enzyme in Suc metabolism and catalyzes the reversible conversion of Suc and UDP to UDP-glucose and fructose, increased in antiAtGLR1.1 lines. A light-regulated basic domain/leucine zipper TF (ATB2/AtbZIP11 At1g75390), which is stimulated by exogenous Suc , increased in antiAtGLR1.1 lines. In addition, the sugar-porter family protein 1 (SFP1 At5g27350) and SFP-members (At3g05400, At3g05160) were significantly elevated in antiAtGLR1.1 plants.

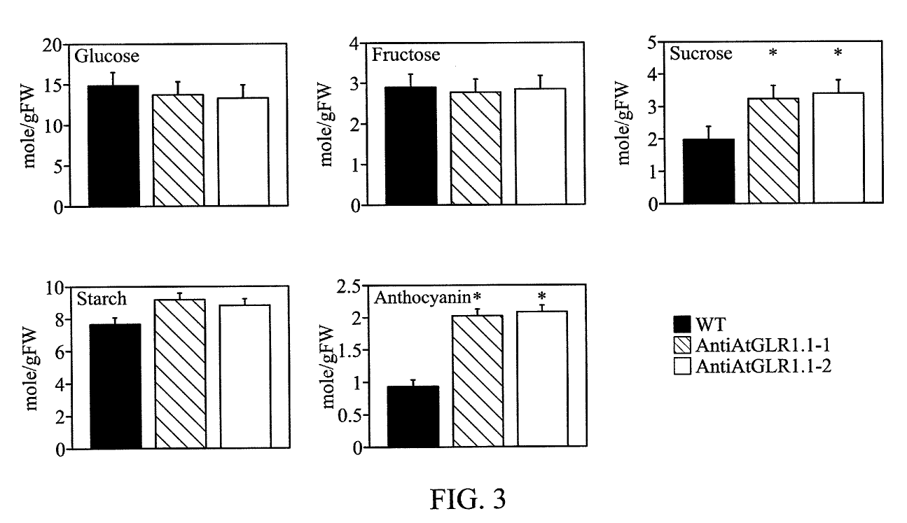

To determine if carbon metabolism was actually altered in antiAtGLR1.1 lines endogenous levels of several sugars and starch were measured in leaves of plants grown (FIG. 3). The Suc content in antiAtGLR1.1 plants significantly increased approximately 68% relative to that in WT. There was also a small (10 to 15%) but reproducible increase in starch in the antiAtGLR1.1 versus WT plants. The levels of glucose (Glc) and fructose (Fru) were unchanged in leaves of antiAtGLR1.1 versus WT plants.

### Carbon Metabolism and Redistribution; Lignin and Cell Wall Biosyntheses

Genes whose products synthesize or modify cell wall components such as cellulose, lignin, pectin, and structural proteins were significantly elevated in antiAtGLR1.1 plants (Table 2), they include; cellulose synthase-like proteins (At4g16590, At4g23990, At5g17420), putative pectinesterases (At2g43050, At3g10720, At3g59010, At4g33220), putative arabinogalactan-proteins (At1g68725, At5g60490, At5g10430, At5g03170). Several genes associated with lignin biosynthesis were significantly up-regulated in the antiAtGLR1.1 lines, these include, cytochrome P450 coumarate to p-Coumaryl-CoA (At1g74540), a putative S-adenosyl-L-methionine: trans-caffeoyl-Coenzyme A 3-O methyltransferase (CCOMTL2, At1g67990), two cinnamyl alcohol dehydrogenases (CAD, At4g34230, At1g09500), prephenate dehydratase (PrD, At5g22630), kynurenine aminotransferase/glutamine transaminase (KAT/GT, At1g77670), several laccases (At2g38080, At2g29130, At2g40370, At3g09220, At5g05390, At5g60020), peroxidase (At5g42180 At5g05340) and putative dirigent genes (DIR11 At2g22900, DIR6 At4g23690).

There was an increase in a transcription factor, MYB61, that has been shown to be is necessary and sufficient for the remobilization of carbon into lignin biosynthesis. MYB20 (At1g66230), which was shown to have an expression profile that clustered with lignin biosynthetic genes (Ehlting et al. 2005. Plant J. 42: 618-640), is significantly less in the antiAtGLR1.1 plants than in WT. Although Dubos et al. (Dubos et al. 2005. Plant J. 43:348-355) proposed that AtGLRs, through activation of an agonist, may function to negatively modulate endogenous sugar signals that redistribute carbon metabolites into lignin synthesis via a MYB61-mediated pathway, that work was conducted in an vacuolar ATPase deficient mutant called det3 in dark grown seedlings. Furthermore, Dubos et al. (Dubos et al. 2005. Plant J. 43:348-355) showed the general involvement of AtGLRs and did not demonstrate utility of a specific AtGLR in plants grown in the light. This invention specifically demonstrates how to use AtGLR1.1 to control that signaling network to reallocate, redistribute or transport carbon into lignin biosynthesis, since antiAtGLR1.1 lines have high levels of Suc and a transcript profile consistent of an activated MYB61-mediated lignin biosynthetic pathway.

C Metabolism; Fatty Acid and Lipid Metabolism and Redistribution

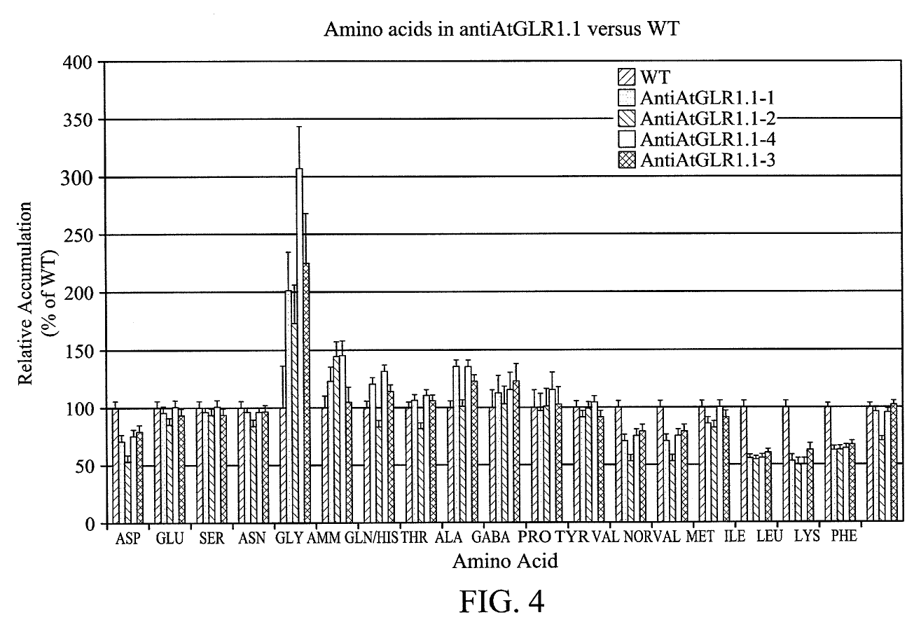

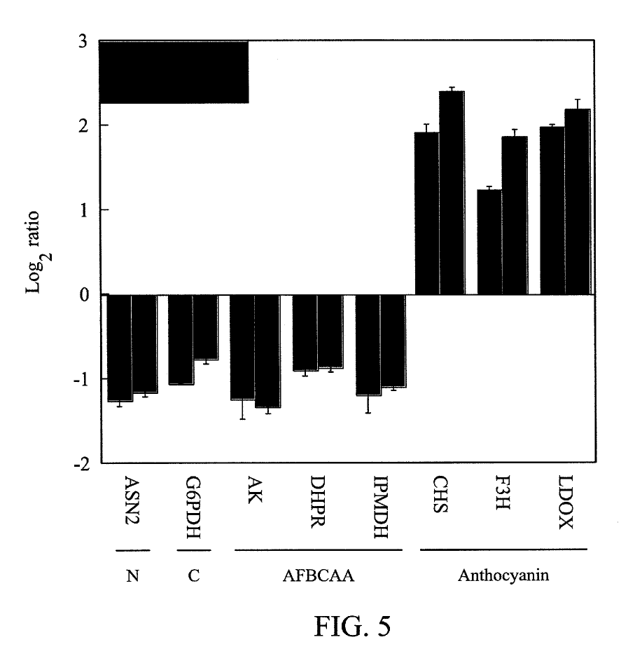

In the antiAtGLR1.1 lines there is a shift of carbon from oxaloacetate (OAA) metabolism and amino acid biosynthesis into lipid metabolism. This is evidenced in two sets of data the (i) decrease in Asp and other Asp derived amino acids and (ii) increase in genes associated with lipid metabolism and mobilization. Free Asp titers in antiAtGLR1.1 lines decreased an average of 33%, relative to WT; this decrease coincides with the reduction in AAT2 transcript and peptide that we previously reported in the antiAtGLR1.1 lines. This finding is consistent with reports that show AAT2, a cytosolic isoform, is the major source of free Asp synthesized in leaves (Schultz et al. 1998. Genet. 149: 491-499). There were significant decreases in the Asp-derived amino acids, Ile and Lys with mean reductions of 43% and 35%, respectively (FIG. 4). The levels of two other amino acids significantly decreased in antiAtGLR1.1 lines relative to WT are Leu, which dropped an average of 45%, and Val, which decreased by an average of 27%. The decrease in these amino acids could be attributed to the drop in Ile, since synthesis of Ile and Val share four common catalytic steps, and Leu is produced from an intermediate of Val synthesis. Consistent with this findings of the amino acid levels several Lys biosynthetic genes in the Asp-derived amino acids, a putative Lys-sensitive Asp kinase (AK At3g02020) and dihydrodipicolinate reductase (DHPR At5g52100), significantly decreased in antiAtGLR1.1 plants. Higher plants synthesize Lys from Asp. Asp is synthesized by the transfer of the amino group from Glu to Asp by asparate aminotransferase (AAT) or from Asn via asparaginase . Asp kinase, the first enzyme in the AFBCAA pathway, is a key enzyme that regulates Lys synthesis. Two 3-isopropylmalate dehydrogenases (IPMDH, At5g14200, At1g31180), Leu biosynthetic genes, were significantly decreased in antiAtGLR1.1 lines. In bacteria, IPMDH catalyzes the oxidative decarboxylation of 3-isopropylmalate to 2 ketoisocaproic acid. Evidence for the role of IPMDH in the control of Leu biosynthesis in higher plants has not been reported but this data strongly suggests that IPMDH plays a key role in Leu biosynthesis, as it does in yeast and E. coli. The expression profiling data was validated by qRT-PCR (FIG. 5), on AK (At3g02020), DHPR (At5g52100) and IPMDH (At5g14200). In agreement with the expression profiling data), the transcripts significantly decreased in all of the antiAtGLR1.1 versus WT lines.

The relocation of OAA for amino acids into lipid metabolism can be explained by the significant decrease in phosphoenolpyruvate carboxylase kinase (PPCK1; At1g08650) and the increased accumulation of two pyruvate decarboxylase (PDC, At5g01320, At4g33070) transcripts. PPCK1 phosphorylates phosphoenolpyruvate carboxylase (PEPC) that catalyzes the synthesis of OAA from phosphoenolpyruvate (PEP) and bicarbonate. PEPC activity is deregulated by phosphorylation. The constitutive over-expression of a positive dominant PEPCK construct in Arabidopsis redirected carbon from sugars into organic acids and amino acids. Therefore the significant decrease in PPCK1 transcript is consistent with the observed decrease in specific the aspartate amino acids and the reallocation or redistribution of carbon into lipid metabolism. The significant increase in two PDCs, the committal steps to alcohol or lipid biosynthesis, transcripts lend further support to redistribution of carbon. Although there is evidence that some of the carbon in antiAtGLR1.1 plants may be diverted into ethanol production, based on the increased accumulation of one alcohol dehydrogenase (ADH, At3g42960) transcript. However, based on the significant increased accumulation of a large number of transcripts associated with lipid metabolism and transport, the carbon is being allocated, distributed or partition diverted from PEPC is channeled into lipid metabolism in antiAtGLR1.1 lines. There are significant changes in the accumulation of the following lipid associated transcripts two putative beta-ketoacyl-CoA synthase (At2g46720, At3g10280) and four genes for putative lipid transfer protein (LTP4; At5g59310, LTP6; At3g08770, At3g51590, At2g44300). Other genes that are associated with lipid metabolism, catabolism or transport that significantly increase in the antiAtGLR1.1 lines are a plastid-lipid associated protein PAP (At1g51110), GDSL-motif lipases/hydrolases (At2g31540, At1g29660, At3g04290, At1g74460, At1g28570, At2g42990, At2g23540), esterase/lipase/thioesterase proteins (At1g08310, At1g54570), and extracellular lipase 6 (EXL6; At1g75930).

The plant hormone jasmonic acid (JA) is derived from lipids. In the antiAtGLR1.1 lines, eight transcripts involved in JA biosynthesis significantly increased in antiAtGLR1.1 lines. These include a pathogen-responsive alpha-dioxygenase (At3g01420), AOS1 (At5g42650), four allene oxide cyclases (At3g25780, At3g25770, At3g25760, At1g13280), OPR1 (At1g76680) and OPR2 (At1g76690).

### Carbon Mediated Anthocyanin Biosynthesis

Anthocyanins are pigmented flavonoids that are predominantly synthesized in the upper epidermis in response to various environmental stresses including light and nutrient deficiency. There was a significant increase in the transcripts for anthocyanin biosynthetic genes such as CHS (At5g13930), F3H (At3g51240) and LDOX (At4g22880) in antiAtGLR1.1 lines. The accumulation of these three anthocyanin biosynthetic genes were validated by qRT-PCR (FIG. 5), in plants grown independently of the microarray experiments, but under the identical conditions.

The microarray and qRT-PCR data were highly correlated. CHS catalyzes the committal step in the pathway leading to the synthesis of anthocyanin. The subsequent steps include a conversion of naringenin chalcone to naringenin by CHI, and hydroxylations of niringenin by F3H and flavonoid 3′ hydroxylase (F3′H). NADPH-dependent DFR leads to the production of leucoanthocyanidins, which are the least common intermediates in anthocyanin and proanthocyanidin biosynthesis. There were observed increases in several genes involved in flavonoid biosynthetic pathways including two chalcone isomerases (At3g55120, At5g5270) and a flavonoid synthase (FLS, At5g08640).

Several anthocyanin biosynthetic genes are regulated by distinct MYB TFs, such as PAP1, PAP2 and TRANSPARENT TESTA 2 (TT2 formerly MYB123. The transcripts of MYB-related TFs, PAP1 (At1g56650) and PAP2 (At1g66390) significantly increased, by approximately 5.5 and 3.2-fold (+2.77 and +1.6 log2/ratio), respectively, in the two independently transformed antiAtGLR1.1 lines. The accumulation of several transcripts in antiAtGLR1.1 lines may be explained by the elevation in PAP1, namely two chalcone isomerases (At3g55120, At5g05270), an anthocyanin 5-0-glucosyltransferase (5GT At4g14090) and a glutathione S-transferase (GST; At5g17220). Conversely, overexpression of PAP1 resulted in decreases in a raffinose synthase family glycosyl hydrolase (RSFGH, At5g20250) and gibberellin-regulated protein 1 (GASA1, At1g75750), thus the decreased accumulation of these latter transcripts in the antiAtGLR1.1 plants might be explained by the increased expression of PAP1.

Total anthocyanins were determined in antiAtGLR1.1 plants to test if there was a corresponding increase in the metabolites, total anthocyanins increased (200%) in antiAtGLR1.1 lines when compared with WT (FIG. 3). Increased expression of anthocyanin biosynthetic genes and increased total anthocyanins in antiAtGLR1.1.I plants are consistent with results from other studies that show that elevated levels of endogenous Suc combined result in increased anthocyanins (Deikman and Hammer, 1995 Plant Physiol. 108: 47-57; Martin et al., 2002; Mita et al., 1997. Plant J. 11: 841-851; Tsukaya et al. 1991. Plant Physiol. 97: 1414-1421; Nikiforova et al. 2003. Plant J. 33: 633-650).

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

TABLE 1
Summary of transcript accumulation in two independently transformed antiAtGLR1.1
lines compared with wild-type in leaves of 30-day-old plants.

| | | | |
|---|---|---|---|
| | | | Average |
| Gene Family | | | accumulation |
| or Pathway | Gene name | Locus ID | (log₂/ratio) |

Carbon metabolism

| | | | |
|---|---|---|---|
| C metabolism-Suc | Sus | At5g20830 | +0.80 |
| C metabolism-Suc | ATB2/AtbZIP1¹ | At1g75390 | +1.26 |
| C metabolism-Suc | SFP1* | At5g27350 | +1.00 |
| C metabolism-Suc | SFP-member | At3g05400 | +2.86 |
| C metabolism-Suc | SFP-member | At3g05160 | +0.67 |
| cell wall | cellulose synthase | At4g16590 | +1.70 |
| cell wall | cellulose synthase | At4g23990 | +2.35 |
| cell wall | cellulose synthase | At5g17420 | +1.24 |
| cell wall | pectinesterase | At2g43050 | +1.48 |
| cell wall | pectinesterase | At3g10720 | −0.91 |
| cell wall | pectinesterase | At3g59010 | +1.12 |
| cell wall | pectinesterase | At4g33220 | −0.74 |
| cell wall | arabinogalactan-protein | At1g68725 | +0.76 |
| cell wall | arabinogalactan-protein | At5g60490 | +2.04 |
| cell wall | arabinogalactan-protein | At5g10430 | +1.03 |
| cell wall | arabinogalactan-protein | At5g03170 | +1.51 |

Lignin biosynthesis and regulation

| | | | |
|---|---|---|---|
| lignin | cytochrome P450 | At1g74540 | +1.21 |
| lignin | CCOMTL2 | At1g67990 | +1.62 |
| lignin | CAD | At4g34230 | +0.68 |
| lignin | CAD | At1g09500 | +0.64 |
| lignin | PrD | At5g22630 | +0.66 |
| lignin | KAT/GT | At1g77670 | +0.69 |
| lignin | laccase | At2g38080 | +1.20 |
| lignin | laccase | At2g29130 | +0.86 |
| lignin | laccase | At2g40370 | +1.20 |
| lignin | laccase | At3g09220 | +0.60 |
| lignin | laccase | At5g05390 | +1.01 |
| lignin | laccase | At5g60020 | +0.68 |
| lignin | peroxidase | At5g42180 | +2.89 |
| lignin | peroxidase | At5g05340 | +0.90 |
| lignin | DIR11 | At2g22900 | +0.67 |
| lignin | DIR6 | At4g23690 | +1.16 |
| lignin | MYB61 | At1g09540 | +0.97 |
| lignin | MYB20 | At1g66230 | −0.95 |

Lipid (oil) biosynthesis and mobilization

| | | | |
|---|---|---|---|
| Lipid regulation | PPCK1 | At1g08650 | −0.79 |
| Lipid regulation | PDC | At5g01320 | +0.86 |
| Lipid regulation | PDC | At4g33070 | +0.76 |
| Lipid metabolism | -ketoacyl-CoA synthase | At2g46720 | +0.83 |
| Lipid metabolism | -ketoacyl-CoA synthase | At3gl0280 | +0.82 |
| Lipid transport | lipid transfer protein, LTP4 | At5g59310 | +2.97 |
| Lipid transport | lipid transfer protein, LTP6 | At3g08770 | +0.73 |
| Lipid transport | lipid transfer protein | At3g51590 | +2.86 |
| Lipid transport | lipid transfer protein | At2g44300 | +0.68 |
| Lipid | plastid-lipid assoc. protein | At1g51110 | +0.85 |
| Lipid | GDSL-motif lipase/hydrolase | At2g31540 | +0.91 |
| Lipid | GDSL-motif lipase/hydrolase | At1g29660 | +0.85 |
| Lipid | GDSL-motif lipase/hydrolase | At3g04290 | +0.69 |
| Lipid | GDSL-motif lipase/hydrolase | At1g74460 | +0.83 |
| Lipid | GDSL-motif lipase/hydrolase | At1g28570 | +0.83 |
| Lipid | GDSL-motif lipase/hydrolase | At2g42990 | +1.15 |
| Lipid | GDSL-motif lipase/hydrolase | At2g23540 | +1.12 |
| Lipid | esterase/lipase/thioesterase | At1g08310 | −1.04 |
| Lipid | esterase/lipase/thioesterase | At1g54570 | +0.96 |
| Lipid | extracellular lipase 6, EXL6 | At1g75930 | +1.93 |
| Lipid, JA | allene oxide cyclase | At3g25770 | +1.78 |
| Lipid, JA | allene oxide cyclase | At3g25760 | +1.21 |
| Lipid, JA | allene oxide cyclase | At3g25780 | +1.19 |
| Lipid, JA | OPR2 | At1g76690 | +0.81 |
| Lipid, JA | allene oxide cyclase | At1g13280 | +0.78 |
| Lipid, JA | path.-respon. -dioxygenase | At3g01420 | +0.97 |
| Lipid, JA | OPR1 | At1g76680 | +0.87 |

Anthocyanin and flavonoid?? biosynthesis

| | | | |
|---|---|---|---|
| anthocyanin | CHS | At5g13930 | +2.20 |
| anthocyanin | F3H | At3g51240 | +1.56 |
| anthocyanin | LDOX | At4g22880 | +1.49 |
| anthocyanin | PAP1 | At1g56650 | +2.77 |
| anthocyanin | PAP2 | At1g66390 | +1.60 |
| anthocyanin-related | chalcone isomerase | At3g55120 | +0.64 |
| anthocyanin-related | chalcone isomerase | At5g05270 | +0.88 |
| anthocyanin-related | FLS | At5g08640 | +1.70 |
| anthocyanin-related | 5GT | At4g14090 | +1.20 |
| anthocyanin-related | GST | At5g17220 | +1.85 |
| anthocyanin-related | RSFGH | At5g20250 | −0.61 |
| anthocyanin-related | GASA1 | At1g75750 | −0.61 |

¹Abbreviated as in the text.

We claim:

1. A method of delivering to a plant target cell a DNA that is expressed in the target cell comprising administering a vector to the target cell, wherein said vector transduces the target cell; and wherein said vector has been modified to comprise a DNA which comprises an expressible gene and said gene is expressed in said target cell either constitutively or under regulatable conditions, and wherein the expressible gene encodes a messenger RNA which is antisense with respect to a messenger RNA transcribed from a gene endogenous to said cell, and wherein the antisense RNA is antisense to the mRNA which is translatable into a glutamate receptor.

2. The method of claim 1, wherein the glutamate receptor is Arabidopsis thaliana glutamate receptor 1.1 (AtGLR1.1).

3. A method of regulating plant metabolism, comprising: delivering to a plant target cell a DNA that is expressed in the target cell comprising administering a vector to the target cell, wherein said vector transduces the target cell; and wherein said vector has been modified to comprise a DNA which comprises an expressible gene and said gene is expressed in said target cell either constitutively or under regulatable conditions, and wherein the expressible gene encodes a messenger RNA which is antisense with respect to a messenger RNA transcribed from a gene endogenous to said cell, and wherein the antisense RNA is antisense to the mRNA which is translatable into a glutamate receptor.

4. The method of claim 3, wherein the antisense RNA is antiGLR and is capable of altering carbon-based signaling molecules, growth regulators, structural compounds to control and improve plant growth, development, yield and crop quality.

5. The method of claim 3, wherein the antisense RNA is antiGLR and is capable of reallocating carbon metabolites in higher plants and altering the accumulation or distribution of carbon metabolites or compounds, wherein said metabolites or compounds comprise sugars, organic acids, carbohydrates, starch, oils, lipids, callose, cellulose, hemicellulose and secondary compounds including anthocyanins, flavonoids and lignins.

6. The method of claim 3, wherein the glutamate receptor is Arabidopsis thaliana glutamate receptor 1.1 (AtGLR1.1).

7. A DNA vector comprising an expressible gene that is expressed in a target plant cell, wherein the expressible gene encodes a messenger RNA which is antisense with respect to a messenger RNA transcribed from a gene endogenous to said cell, and wherein the antisense RNA is antisense to the mRNA which is translatable into a glutamate receptor.

8. A plant comprising the DNA vector of claim 7.
